(19) United States
(12) Patent Application Publication
(10) Pub. No.: US 2002/0133070 A1
(43) Pub. Date: Sep. 19, 2002

(54) Method for performing magnetic resonance angiography with subtraction of projection images

(76) Inventors: Yuexi Huang, Toronto (CA); Graham A. Wright, Toronto (CA)

Correspondence Address: Barry E. Sammons, Quarles and Brady LLP, 411 East Wisconsin Avenue, Milwaukee, WI, 53202, US

(21) Appl. No.: 09/761,979

(22) Filed: Jan. 17, 2001

(51) Int. Cl. A61B 5/05

(52) U.S. Cl. 600/420

(57) ABSTRACT

An MRI system is employed to acquire a 3D image which is enhanced by injection of a contrast agent into the subject's vasculature. A 3D mask image is also acquired and 2D projection images are produced from both 3D images. The resulting 2D projection mask image is subtracted from the 2D enhanced projection image to produce the contrast enhanced MRA image.

### BACKGROUND OF THE INVENTION

The field of the invention is magnetic resonance angiography (“MRA”), and particularly, studies of the human vasculature using contrast agents which enhance the NMR signals.

Diagnostic studies of the human vasculature have many medical applications. X-ray imaging methods such as digital subtraction angiography (“DSA”) have found wide use in the visualization of the cardiovascular system, including the heart and associated blood vessels. One of the advantages of these x-ray techniques is that image data can be acquired at a high rate (i.e. high temporal resolution) so that a sequence of images may be acquired during injection of the contrast agent. Such “dynamic studies” enable one to select the image in which the bolus of contrast agent is flowing through the vasculature of interest. Images showing the circulation of blood in the arteries and veins of the kidneys, the neck and head, the extremities and other organs have immense diagnostic utility. Unfortunately, however, these x-ray methods subject the patient to potentially harmful ionizing radiation and often require the use of an invasive catheter to inject a contrast agent into the vasculature to be imaged. There is also the issue of increased nephro-toxicity and allergic reactions to iodinated contrast agents used in conventional x-ray angiography.

Magnetic resonance angiography (MRA) uses the nuclear magnetic resonance (NMR) phenomenon to produce images of the human vasculature. When a substance such as human tissue is subjected to a uniform magnetic field (polarizing field BO), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited spins, and after the excitation signal B₁ is terminated, this signal may be received and processed to form an image.

When utilizing these signals to produce images, magnetic field gradients (Gₓ, G_{y} and G_{z}) are employed. Typically, the region to be imaged is scanned by a sequence of measurement cycles in which these gradients vary according to the particular localization method being used. The resulting set of received NMR signals are digitized and processed to reconstruct the image using one of many well known reconstruction techniques.

MR angiography (MRA) has been an active area of research. Two basic techniques have been proposed and evaluated. The first class, time-of-flight (TOF) techniques, consists of methods which use the motion of the blood relative to the surrounding tissue. The most common approach is to exploit the differences in magnetization saturation that exist between flowing blood and stationary tissue. Flowing blood, which is moving through the excited section, is continually refreshed by spins experiencing fewer excitation pulses and is, therefore, less saturated. The result is the desired image contrast between the high-signal moving blood and the low-signal stationary tissues.

MRA methods have also been developed that encode motion into the phase of the acquired signal as disclosed in U.S. Pat. No. Re. 32,701. These form the second class of MRA techniques and are known as phase contrast (PC) methods. Currently, most PC MRA techniques acquire two images, with each image having a different sensitivity to the same velocity component. Angiographic images are then obtained by forming either the phase difference or complex difference between the pair of velocity-encoded images.

To enhance the diagnostic capability of MRA a contrast agent such as gadolinium can be injected into the patient prior to the MRA scan. Excellent diagnostic images may be acquired using contrast-enhanced MRA if the data acquisition is properly timed with the bolus passage.

While vascular images may be produced simply by selecting a set of data points located in a cross section through the reconstructed 3D image, such images have limited diagnostic value. This is because blood vessels usually do not lie in a single plane and such cross sectional images show only short pieces or cross sections of many vessels that happen to pass through the selected plane. Such images are useful when a specific location in a specific vessel is to be examined, but they are less useful as a means for examining the health of the vascular system and identifying regions that may be diseased.

For assessing overall blood vessel structure and health it is more useful to project the 3D array of NMR image data into a single 2D projection image to produce an angiogram-like picture of the vascular system. The most commonly used technique for doing this is to project a ray from each pixel in the 2D projection image through the array of data points and select the data point which has the maximum value. The value selected for each ray is used to control the brightness of its corresponding pixel in the 2D projection image. This method, referred to as the “maximum intensity pixel” or “MIP” technique, is very easy to implement and it gives aesthetically pleasing images.

The non-invasiveness of MRA makes it a valuable screening tool for cardiovascular diseases. Screening typically requires imaging vessels in a large volume. This is particularly true for diseases in the runoff vessels of the lower extremity. The field of view (FOV) in MR imaging is limited by the volume of the B₀ field homogeneity and the receiver coil size (typically, the FOV<48 cm on current commercial MR scanners). The anatomic region of interest in the lower extremity, for example, is about 100 cm and this requires several FOVs, or stations, for a complete study.

There are two approaches used to acquire 3D images from multiple fields of view. One approach is to make a single injection of contrast agent and move the patient table through a series of stations to follow, or “chase” the bolus through the vasculature to be imaged. This method is described, for example, in U.S. Pat. Nos. 5,298,148 and 5,924,987.

The alternative approach is to move the patient to multiple stations and inject contrast agent at each station. Due to the time delay between multiple injections, the contrast from earlier injections flows into the veins and the veins are also visible in subsequent images. The arteries and veins may overlap in reconstructed MIP images, making diagnosis difficult. To minimize this problem it is common to acquire a mask image prior to contrast injection at each station and to subtract the mask image from the contrast enhanced image.

Several methods are used for combining the processes of mask subtraction and MIP projection. The most common approach is to subtract the magnitude images of the two 3D data sets slice by slice and then do the MIP projection on the resulting 3D difference image. Although conspicuity increases somewhat after subtraction, the difference often does not translate into any measurable diagnostic gain. Complex subtraction (subtract the complex raw data of the two 3D data sets slice by slice then do MIP) was proposed to reduce the partial volume effect in U.S. Pat. No. 5,827,187. However, unlike the 2D thick slab protocol used therein, in 3D high resolution MR angiography, the image pixels are about 1 mm, less than the diameter of main arteries, and the partial volume effect is not significant.

### SUMMARY OF THE INVENTION

The present invention is a method and apparatus for producing a 2D projection image from a 3D image data set from which a 3D mask image is subtracted. More particularly, the invention includes acquiring and reconstructing a 3D image, acquiring and reconstructing a 3D mask image, producing a 2D projection image at a selected projection angle through the 3D image, producing a 2D projection mask image at the selected projection angle through the 3D mask image, and subtracting the 2D projection mask image from the 2D projection image.

We have discovered that by performing, for example, a maximum intensity projection separately on the acquired 3D image and the acquired 3D image mask, and then performing the subtraction of the mask, improved signal-to-noise ratio (SNR) and arterial conspicuity is achieved in the resulting 2D projection image, compared to other subtraction techniques. This results in improved visualization of small vessels, especially in low SNR acquisitions.

### BRIEF DESCRIPTION OF THE DRAWINGS

FIG. 1 is a block diagram of an MRI system which employs the present invention;

FIG. 2 is a graphic representation of a pulse sequence performed by the MRI system of FIG. 1 to practice a preferred embodiment of the invention;

FIG. 3 is a pictorial representation of a patient illustrating a region of interest comprised of three overlapping fields of view; and

FIG. 4 is a flow chart illustrating the steps performed using the MRI system of FIG. 1 to practice the preferred embodiment of the invention.

### DESCRIPTION OF THE PREFERRED EMBODIMENT

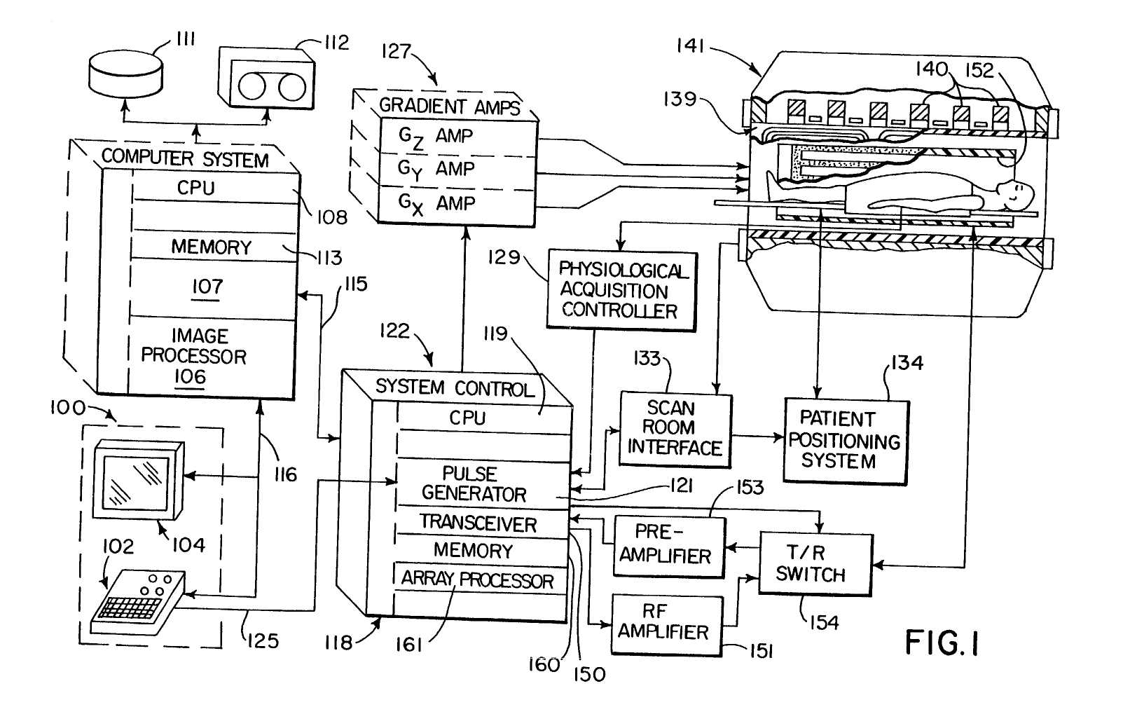

Referring first to FIG. 1, there is shown the major components of a preferred MRI system which incorporates the present invention. The operation of the system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules which communicate with each other through a backplane. These include an image processor module 106, a CPU module 108 and a memory module 113, known in the art as a frame buffer for storing image data arrays. The computer system 107 is linked to a disk storage 111 and a tape drive 112 for storage of image data and programs, and it communicates with a separate system control 122 through a high speed serial link 115.

The system control 122 includes a set of modules connected together by a backplane. These include a CPU module 119 and a pulse generator module 121 which connects to the operator console 100 through a serial link 125. It is through this link 125 that the system control 122 receives commands from the operator which indicate the scan sequence that is to be performed. The pulse generator module 121 operates the system components to carry out the desired scan sequence. It produces data which indicates the timing, strength and shape of the RF pulses which are to be produced, and the timing of and length of the data acquisition window. The pulse generator module 121 connects to a set of gradient amplifiers 127, to indicate the timing and shape of the gradient pulses to be produced during the scan. The pulse generator module 121 also receives patient data from a physiological acquisition controller 129 that receives signals from a number of different sensors connected to the patient, such as ECG signals from electrodes or respiratory signals from a bellows. And finally, the pulse generator module 121 connects to a scan room interface circuit 133 which receives signals from various sensors associated with the condition of the patient and the magnet system. It is also through the scan room interface circuit 133 that a patient positioning system 134 receives commands from the pulse generator module 121 to move the patient to desired positions. The operator can thus control the operation of the patient positioning system 134 through the keyboard and control panel 102.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a whole-body RF coil 152. A transceiver module 150 in the system control 122 produces pulses which are amplified by an RF amplifier 151 and coupled to the RF coil 152 by a transmit/receive switch 154. The resulting signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150.

The transmit/receive switch 154 is controlled by a signal from the pulse generator module 121 to electrically connect the RF amplifier 151 to the coil 152 during the transmit mode and to connect the preamplifier 153 during the receive mode. The transmivreceive switch 154 also enables a separate RF local coil to be used during the receive mode.

The NMR signals picked up by the RF local coil are digitized by the transceiver module 150 and transferred to a memory module 160 in the system control 122. When the scan is completed and an entire array of data has been acquired in the memory module 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 in accordance with the teachings of the present invention and conveyed to the operator console 100 and presented on the display 104.

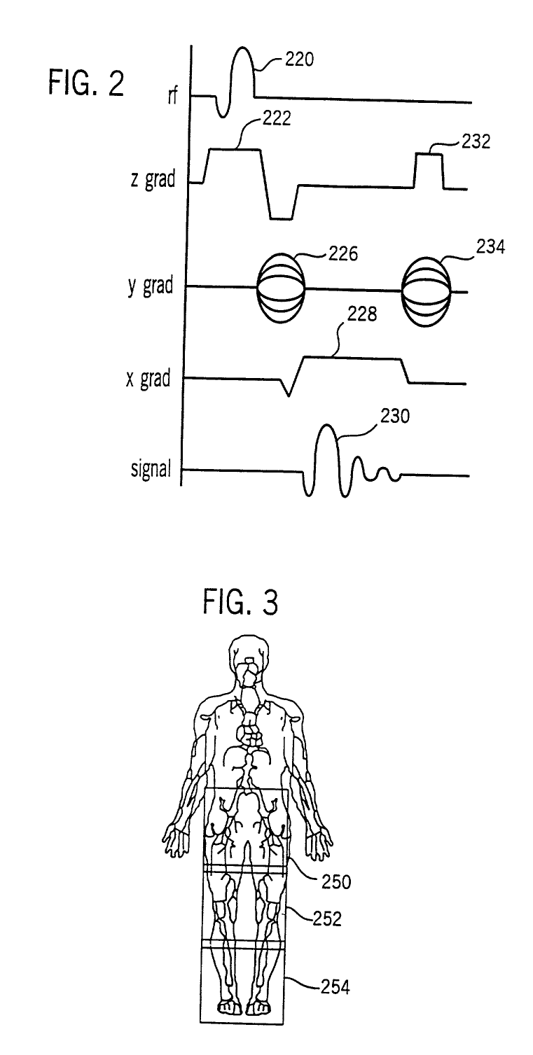

While many pulse sequences may be used to practice the present invention, in the preferred embodiment a 3D gradient-recalled echo pulse sequence is used to acquire the NMR data. Referring particularly to FIG. 2, an RF excitation pulse 220 having a flip angle of 200 is produced in the presence of a slab select gradient pulse 222 to produce transverse magnetization in the 3D volume of interest as taught in U.S. Pat. No. 4,431,968. This is followed by a phase encoding gradient pulse 224 directed along the z axis and a phase encoding gradient pulse 226 directed along the y axis. A readout gradient pulse 228 directed along the x axis follows and a NMR signal 230 is acquired and digitized as described above. After the acquisition, rewinder gradient pulses 232 and 234 are applied to rephase the magnetization before the pulse sequence is repeated as taught in U.S. Pat. No. 4,665,365.

As is well known in the art, the pulse sequence is repeated and the phase encoding pulses 224 and 226 are stepped through a series of values to sample the 3D k-space in the field of view. In the preferred embodiment 64 phase encodings are employed along the z axis and 256 phase encodings are employed along the y axis. Sampling along the kₓ axis is performed by sampling the echo signal 230 in the presence of the readout gradient pulse 228 during each pulse sequence. It will be understood by those skilled in the art that only a partial sampling along the kₓ axis is performed and the missing data is computed using a homodyne reconstruction or by zero filling. This enables the echo time (TE) of the pulse sequence to be shortened to 1.5 ms and the pulse repetition rate (TR) to be shortened to 9.5 ms.

Referring to FIG. 3, an examination of the vasculature of a patient's legs can be performed by dividing up the region of interest into a plurality of overlapping fields of view indicated at 250, 252 and 254. This is accomplished by moving a patient table 256 to three successive locations, or stations, within the bore of the magnet to align the respective centers of the field of view with the isocenter of the MRI system.

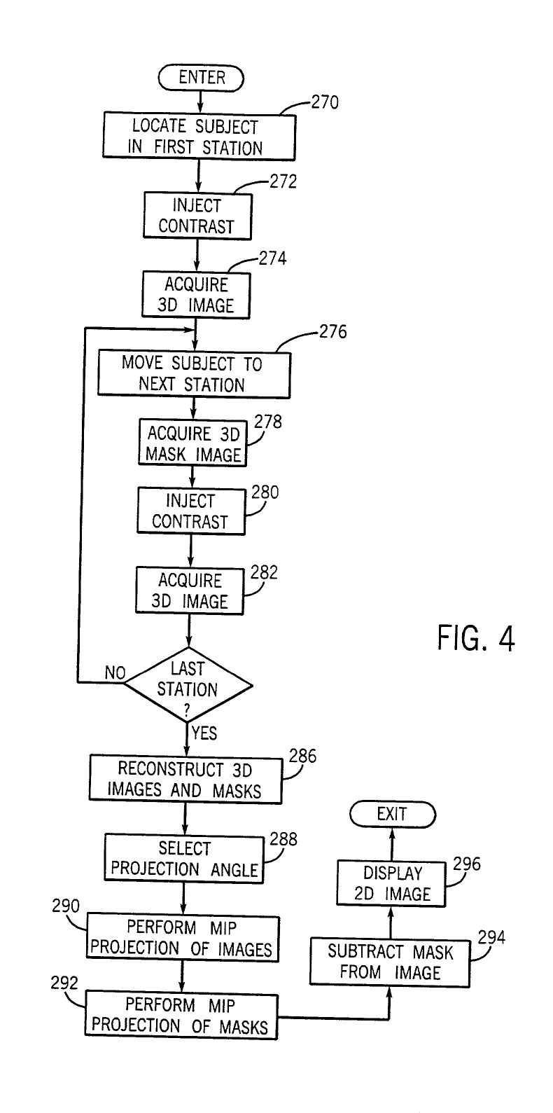

Referring particularly to FIG. 4, in the preferred embodiment of the invention the patient is placed in the MRI system such that the lower FOV 254 is aligned with the system isocenter as indicated at process block 270. The contrast agent is then injected at process block 272, which in the preferred embodiment is 20 cc each of a contrast agent such as that sold under the trademark “Magnevist” by Berlex and a saline solution at a flow rate of 1.5 ml/s. After a suitable time delay to enable the contrast to arrive in the FOV, a 3D image is acquired at process block 274, using the above-described pulse sequence and a centric view order such as that described in U.S. Pat. No. 5,912,557. A 256×256×64 element array of complex k-space data is acquired over a 40×40×6 cm field of view.

The patient is then moved as indicate at process block 276 to position the next FOV in alignment with the MRI system isocenter. A 3D mask image is then acquired as indicated at process block 278 using the same pulse sequence and scan parameters. In this acquisition it is not necessary to use centric view ordering since the objective is to capture a picture of the contrast remaining in the veins and arteries from the previous injection. This is followed by another contrast injection at process block 280 identical to that described above, and image acquisition as indicated at process block 282. The 3D image acquisition employs the same pulse sequence and scan parameters described above, and the centric view order acquisition is timed for bolus arrival in the FOV.

If further FOVs are to be acquired as determined at decision block 284, the system loops back to process block 276 to acquire another 3D mask and corresponding 3D image. Otherwise, the data acquisition phase is completed and the acquired 3D images and corresponding 3D mask are reconstructed at process block 286 using the complex, three-dimensional Fourier transformation discussed above. Magnitude images are then produced from each reconstructed 3D image and 3D mask by calculating the square root of the sum of the squares of the real and imaginary components of the complex elements therein. As a result, each reconstructed 3D image and 3D mask is a 256×256×64 array of voxel intensity values.

The next step is to select a projection angle as indicated at process block 288. In the preferred embodiment this is accomplished by displaying the 3D image to the operator and enabling the operator to revolve the image on the display until the depicted vasculature is viewed from the desired vantage point. A MIP projection of each of the reconstructed 3D images is then performed at process block 290 and a MIP projection of each of the reconstructed 3D mask images is performed to process block 292. As is known in the art, each MIP projection is performed by selecting the maximum values in the 3D image or 3D mask which lie on rays projected therethrough at the selected projection angle. Each such maximum value becomes the intensity value of a corresponding pixel in the 2D projection image. While the MIP projection technique is preferred, other non-linear projection techniques may also be employed.

As indicated at process block 294 each 2D projected mask is then subtracted from its corresponding 2D projected image. The projection images are 2D arrays of pixel intensity values and this subtraction is a straight forward subtraction of each mask pixel from it corresponding image pixel. The result is displayed as indicated at process block 296.

A study was performed on five patients to compare the results when using the present invention (“MIP subtraction”) from the well known “complex subtraction” and “magnitude subtraction” methods. One set of clinical MR DSA MIP images were produced with these three different subtraction methods. The relative background tissue statistics are shown in Table 1 (region selected in the leg muscle). After MIP subtraction, the mean of the background noise is dramatically decreased relative to those for complex and magnitude subtractions. After doing the MIP projection, the mean of the noise distribution gets bigger, while the deviation gets smaller. With MIP subtraction, this bias in the background subtracts out. Meanwhile, for magnitude and complex subtraction, the MIP following subtraction introduces significant bias in the final image.

Table 1. Background tissue statistics in the clinical study. Means and standard deviations of five patient studies were measured separately and were normalized to the mean after the complex subtraction. The ± values represent one standard deviation for each of the reported values.

| | Complex | Magnitude | MIP |
|---|---|---|---|
| | subtraction | subtraction | subtraction |
| Tissue mean | 1 | 0.76 ± 0.02 | 0.23 ± 0.09 |
| Tissue std deviation | 0.16 ± 0.2 | 0.16 ± 0.02 | 0.18 ± 0.08 |

It should be apparent to those skilled in the art that many variations are possible without departing from the spirit of the invention. For example, a number of other projection techniques are known in the art, such as that described in U.S. Pat. No. 5,204,627. Also, there are other clinical applications of the present invention in which a 2D projection difference image is to be produced from an acquired 3D image and 3D mask image, such as the time resolved MRA method described in U.S. Pat. No. 5,713,358.

1. A method for producing a projection difference image from an acquired 3D image and an acquired 3D mask image, the steps comprising:
a) selecting a projection angle;
b) producing a 2D projection image by projecting the acquired 3D image at the selected projection angle;
c) producing a 2D projection mask image by projecting the acquired 3D mask image at the selected projection angle; and
d) producing the projection difference image by subtracting 10 the 2D projection mask image from the 2D projection image.

2. The method as recited in claim 1 in which the projections in steps b) and c) are produced using a maximum intensity pixel projection.

3. The method as recited in claim 1 in which the 3D image and the 3D mask image are each 3D arrays of magnitude values and steps b) and c) are performed by selecting magnitude values from each 3D array.

4. The method as recited in claim 1 in which the 3D image and the 3D mask image are acquired from a subject using a magnetic resonance imaging system.

5. The method as recited in claim 4 in which the 3D image is acquired after the subject is injected with a contrast agent which enhances image contrast.

6. The method as recited in claim 5 in which the contrast agent is injected into vasculature in the subject.

7. The method as recited in claim 1 in which the acquired 3D image and the acquired 3D mask image are each comprised of a three-dimensional array of elements representing magnitude values from the same field of view.

8. The method as recited in claim 7 in which steps b) and c) are performed by selecting the maximum magnitude values lying on projections through the respective 3D image and 3D mask image.

9. A method for performing a scan with a magnetic resonance imaging (MRI) system, the steps comprising:
a) acquiring a 3D mask image of a field of view in a subject placed in the MRI system;
b) injecting a contrast agent in the subject to enhance the NMR signals from the subject's vasculature;
c) acquiring an enhanced 3D image of the field of view in the subject;
d) selecting a projection angle;
e) producing a 2D projection image by projecting the enhanced 3d image at the selected projection angle using a non-linear projection technique;
f) producing a 2D projection mask image by projecting the 3D mask image at the selected projection angle using the non-linear projection technique; and
g) producing a projection difference image by subtracting the 2D projection mask image from the 2D projection image.

10. The method as recited in claim 9 which includes:
g) moving the subject; and
h) producing a second projection difference image of another field of view by repeating steps a), b), c), e), f), and g).

11. The method as recited in claim 9 in which steps a) and c) each include:
i) performing a series of pulse sequences with the MRI system to acquire a three-dimensional array of complex k-space data;
ii) Fourier transforming the array of complex k-space data to form a three-dimensional array of complex image data; and
iii) calculating the magnitude of each complex value in the three-dimensional array of complex image data.

12. The method as recited in claim 11 in which steps e) and f) are performed by selecting the maximum magnitude values lying on projections through the respective enhanced 3D image and 3D mask image.

13. The method as recited in claim 9 in which the projections in steps e) and f) are produced using a maximum intensity pixel projection.

14. The method as recited in claim 11 in which the same pulse sequence is employed to acquire both three-dimensional arrays of k-space data.

15. The method as recited in claim14 in which the pulse sequence is a gradient-recalled echo pulse sequence.

16. The method as recited in claim11 in which the pulse sequences performed to acquire the enhanced 3D image are performed in a centric view order.

17. The method as recited in claim 9 in which the step of selecting a projection angle includes:
i) displaying the enhanced 3D image; and
ii) using the displayed enhanced 3D image to indicate a 5 projection angle.
